(19) United States
(12) Patent Application Publication
(10) Pub. No.: US 2022/0331452 A1
(43) Pub. Date: Oct. 20, 2022

(54) IMPROVEMENTS IN OR RELATING TO ORGANIC COMPOUNDS

(71) Applicant: GIVAUDAN SA, Vemier (CH)

(72) Inventors: Ruth C. MOYSEN, Newchurch, Romney Marsh Kent (GB); Thomas BRIERE, Paris (FR); Julia BROOKS, Ashford, Kent (GB); Linda KEHAYA, Ashford, Kent (GB); Ioannis KONTARIS, Ashford, Kent (GB); Kristopher George MAGEE, Bristol (GB); Christopher PLEY-DELL-PEARCE, Bristol (GB)

(21) Appl. No.: 17/619,677

(22) PCT Filed: May 1, 2020
(86) PCT No.: PCT/EP2020/062179

§ 371 Date: Dec. 16, 2021

(30) Foreign Application Priority Data
Jun. 27, 2019 (GB) 1909222.0

(51) Int. Cl. A61K 49/00; A61K 8/9789; A61K 8/33; A61K 8/49; A61K 8/34; A61K 8/35; A61K 8/31; A61Q 11/00

(52) U.S. Cl. CPC A61K 49/0004; A61K 8/9789; A61K 8/33; A61K 8/498; A61K 8/34; A61K 8/35; A61K 8/31; A61K 8/342; A61Q 11/00

(57) ABSTRACT

A method of assessing the impact of a test oral care flavour composition on the well-being of human subjects is provided, as well as a method of preparing or optimizing an oral care flavour composition for enhancing the well-being of a human subject, oral care flavour compositions that enhance the well-being, and consumer products containing them.

### FIELD OF THE INVENTION

The present invention relates to a method of measuring the impact of oral care flavour compositions on the well-being of human subjects, to a method of preparing or optimising oral care flavour compositions that enhance well-being, to oral care flavour compositions that enhance well-being, as well as consumer products containing the oral care flavour compositions.

### BACKGROUND OF THE INVENTION

Well-being (or more precisely “subjective well-being”) is a complex concept that generally people cannot articulate straightforwardly. Most people would simply equate well-being with the feeling of happiness, or maybe relaxation. A more precise definition of well-being sometimes used in the academic literature refers to well-being as the affective and cognitive assessment by a person of his or her life. In this definition, the so-called “affective” element refers to a person's moods and emotions. A positive affect arises when the moods and emotions experienced are pleasant (e.g. joy, elation, affection, and the like), whereas affect is deemed to be negative when moods and emotions experienced are unpleasant (e.g. guilt, anger, shame, and the like). Psychologically, a person experiences a mood of happiness when the affective balance is such that the positive affect outweighs negative affect.

The other important element of subjective well-being—the so-called “cognitive” element—refers to a person's evaluation of his or her life satisfaction both in global terms (e.g. life as a whole) and in domain terms, such as in terms of satisfaction in personal relationships, the control one has over one's life, one's perceived sense of purpose, and the like.

Well-being is usually measured using self-report methods, typically in the form of questionnaires.

Respondents are asked a series of questions, each relating to a metric relevant to a different subjective state that is considered to have an effect on affective or cognitive elements of well-being. This can include subjective states that have a positive or a negative impact on subjective well-being, e.g. sadness, tiredness, patience, irritation, and the like. For each question, e.g. of the type “are you sad?”, respondents are asked to rate the pertinence of each subjective state on a rating scale between polar extremes, e.g. from not very pertinent to highly pertinent. By subjecting a respondent's response data to statistical analysis it is possible to quantify well-being. In this way, many indices have been developed to measure well-being and related concepts. However, knowing which questions to ask can significantly affect the reliability of such measurement techniques.

Reliable means of measuring or rating subjective well-being are highly desirable owing to the economic, political and sociological significance attached to the well-being parameter since the late 20^{th} century. Well-being, too, has assumed increased importance more recently as a design parameter in the development and commercialization of consumer products, such as laundry care, household care and personal care products. Manufacturers of these products recognize that modern consumers seek emotional experience from the products they purchase, and they are particularly interested in brands to which they feel personally connected and which offer them a sense of health and well-being.

Fragrances and flavours have been widely employed by consumer product companies in order to impart to their products pleasant, well-liked odours and tastes that promote consumer liking and which influence purchasing decisions for this reason. However, in an increasingly competitive market place, mere liking is often not sufficient to differentiate one brand over its competitors. Accordingly, in the market execution of their products, consumer product companies frequently refer to wide-ranging product benefits, typically communicated through diverse advertising campaigns, as well as on the packaging and labelling of their products, which together form an important part of their branding strategy. New differentiating effects are constantly sought, and flavours have often been employed as a means to achieve those effects. For example, flavours have been employed to create real or perceived functional effects that may relate to oral care, such as hygiene effects, malodour-counteracting effects, and the like.

In addition to its functional effects, odour and taste are known to elicit emotional responses in human subjects. The temporary beneficial psychological effects of odour on human emotions have been studied extensively in the academic literature. For example, the effects of odour on the affective (i.e. emotional) experience of human subjects have been studied by C. Chrea et al. (2009), “Mapping the semantic space for the subjective experience of emotional responses to odours”, Chemical Senses, 34 (2009) 49-62 and S. Delplanque et al. “How to map the affective semantic space of Scents”, Cognition & Emotion 26(5) (2012) 885-898. In particular, these papers describe a verbal measurement method in the form of a questionnaire, wherein the measurement terms are selected such that it is possible to differentiate the various moods and emotions evoked by odours emanating from perfumed and flavoured products. However, in these papers, the cognitive aspect of well-being is under-estimated and limited to memory aspects associated with the olfactory function.

The patent literature also describes perfume compositions for use in consumer products that are specifically designed to create pleasant mood states (see for example WO 2008/050086 A1) and quality of sleep (see for example WO 2016/146673 A1). However, all of these prior art references touch on only distinct emotional aspects of well-being (e.g. happiness or quality of sleep), without actually addressing both affective and cognitive aspects of well-being as a holistic concept.

Thus, there remains a need to provide a method of measuring the effect of flavour compositions on the subjective well-being of human subjects, as well as a method of designing flavour compositions that enhance well-being of human subjects. More particularly, there remains a need to provide such methods to measure the effect of oral care flavour compositions on well-being and to design oral care compositions that enhance the well-being of human subjects, particularly upon teeth brushing.

### SUMMARY OF THE INVENTION

In a first aspect, the present invention provides a method of assessing an impact of a test oral care flavour composition on the well-being of human subjects, the method comprising the steps of:
a) Having one or more human subjects assess their well-being before teeth brushing and in the absence of a test oral care flavour composition by scoring a plurality of well-being attributes;
   i) wherein the well-being attributes include “not anxious”, “not sad”, “not restless”, “not frustrated”, “not stressed”, “happy”, “optimistic”, “excited”, “satisfied”, “motivated”, “invigorated”, “interested”, “not bored”, “not fatigued”, “mentally alert”, “calm”, “relaxed”, and “patient”; and
   ii) wherein each well-being attribute has a corresponding weight;
b) Calculating a base well-being score before teeth brushing and in the absence of the test oral care flavour composition by multiplying the score of each well-being attribute by the corresponding weight to form a product and summing these products over all well-being attributes;
c) Repeating steps a) and b) after teeth brushing with a toothpast comprising a test oral care flavour composition in order to determine a well-being score with the test oral care flavour composition; and
d) Determining the impact of the test oral care flavour composition on the well-being by subtracting the base well-being score from the well-being score with the test oral care flavour composition.

In a second aspect, the present invention provides a method of preparing or optimizing an oral care flavour composition for enhancing the well-being of a human subject, the method comprising the steps of:
a-d) Assessing the impact of one or more oral care flavour compositions on the well-being of human subjects according to the method described above; and
e) Selecting an oral care flavour composition with a positive impact on the well-being.

In a third aspect, the present invention provides an oral care flavour composition comprising:
(i) one or more oral care flavour ingredients having an anise organoleptic attribute, wherein the total amount of oral care flavour ingredients having an anise organoleptic attribute is at least 3.0 wt % of the oral care flavour composition, more preferably at least 5.0 wt %, even more preferably at least 7.0 wt %, and most preferably at least 8.0 wt %;
(ii) one or more oral care flavour ingredients having a peppermint organoleptic attribute, wherein the total amount of oral care flavour ingredients having a peppermint organoleptic attribute is at least 50 wt % of the oral care flavour composition, more preferably at least 55 wt %, and most preferably at least 60 wt %; and
(iii) at least one oral care flavour ingredient having another organoleptic attribute, wherein said other organoleptic attribute is selected from the group consisting of eucalyptus, spearmint, wintergreen, herbal, citrus, floral, fruity, cooling, warming, spicy, and sweet.

Said oral care flavour composition may be prepared or optimized according to the method of the present invention.

In a fourth aspect, the invention provides an oral care consumer product comprising the oral care flavour composition of the present invention and a suitable base.

In a fifth aspect, the invention provides the use of an oral care flavour composition according to the present invention to enhance the well-being of human subjects.

### DETAILED DESCRIPTION OF THE INVENTION

In addressing the deficiencies of the prior art, the applicant has carried out extensive studies into the relationship between oral care flavour compositions and the well-being of human subjects, employing measurements of experimental psychology, consumer testing, and statistical analysis.

More particularly, the applicant provides means for reliably assessing the impact of a oral care flavour composition on well-being by means of a questionnaire that breaks-down the concept of well-being into key attributes that have an effect on affective or cognitive elements of well-being. The key attributes are weighted in a manner that reflects the relative importance of each attribute to the holistic concept of well-being.

The method of the present invention is particularly sensitive and is able to detect the perception of difficult to detect oral care flavour performance in relation to well-being, which might otherwise be hidden below the noise of liking or oral care flavour intensity if the oral care flavours were tested using standard consumer testing techniques. In this way, the methods of the present invention offer a useful creation tool to enable creators of oral care flavour compositions to reduce the number of design iterations needed to arrive at a desirable oral care flavour composition.

A well-being metric was defined by applying experimental psychology and unsupervised clustering of verbal attributes of well-being in order to identify the most relevant dimensions to assess well-being. The well-being attributes take into account various aspects of well-being, such as affective, eudaimonic, social, and physical aspects. These aspects may have both affective (emotional) and cognitive (rational) components.

Typical verbal well-being attributes include, for example, “active”, “adventurous”, “angry”, “anxious”, “ashamed”, “bored”, “calm”, “cautious”, “clear-headed”, “comfortable”, “confident”, “content”, “daring”, “depressed”, “disgusted”, “energetic”, “enthusiastic”, “excited”, “fearful”, “frustrated”, “happy”, “healthy”, “impulsive”, “in control of your situation”, “insecure”, “interested”, “irritated”, “invigorated”, “isolated”, “joyful”, “lonely”, “loved”, “mentally alert”, “motivated”, “nervous”, “optimistic”, “patient”, “relaxed”, “rested”, “restless”, “sad”, “safe”, “satisfied”, “self-conscious”, “self-esteem”, “sociable if in a social environment”, “stressed”, “supported”, “surprised”, “threatened”, “tired”, “uneasy”, “vulnerable”, “well-being”, “worthy” or “worried”.

In the course of the studies, 18 verbal attributes were found particularly responsive to change of well-being perception associated with the occurrence of a perfume composition and with changes of perfume quality. These attributes are therefore particularly relevant for investigating how perfumes may influence the well-being of a human subject. These attributes are: “not anxious”, “not bored”, “calm”, “excited”, “not fatigued”, “not frustrated”, “happy”, “interested”, “invigorated”, “mentally alert”, “motivated”, “optimistic”, “patient”, “relaxed”, “restless”, “not sad”, “satisfied”, and “not stressed”. These 18 well-being attributes are referred to as WB-18 attributes hereinafter.

Surprisingly, well-being attributes associated with social aspects, such as “ashamed”, “isolated”, “lonely”, “loved”, “sociable if in a social environment”, “supported”, and “worthy”, were found irrelevant or not applicable in the context of perfume compositions. Hence, the WB-18 attributes account principally for both affective (emotional) and cognitive (rational) aspects of the lone individual. This well-being metrics was applied to the case of oral care flavour compositions without modifications: The weighting of the well-being attributes was determined as follows:
a) Having one or more human subjects assess their well-being before brushing their teeth and in the absence of a test oral care flavour composition by providing a well-being score for each of the WB-18 attributes;
b) Applying a factor analysis, in particular a principal component analysis, on the well-being scores obtained in step a), in order to determine the impact of each well-being attribute on the overall well-being, resulting in M well-being factors;
   i) wherein each of the well-being factors has a variance expressed as a percentage of the sum of the variances of all well-being factors;
   ii) wherein each of the well-being factors comprises a finite number of well-being attributes, having a loading , expressed as a percentage of the sum of the loadings; and
   iii) wherein none of the well-being attributes appears in more than one well-being factor; and
c) Calculating the well-being score in the absence of the test oral care flavour according to Equation 1:

0=Σ, =ΣΣ (Equation 1).

The principal component analysis revealed that:
the well-being attributes “not anxious”, “not sad”, “not restless”, “not frustrated”, “not stressed” are associated with a first well-being factor having a variance;
the well-being attributes “happy”, “optimistic”, “excited”, “satisfied”, variance “motivated”, “invigorated” are associated with a second well-being factor having a variance;
the well-being attributes “interested” and “not bored” are associated with a third well-being factor having a variance
the well-being attributes “not fatigued” and “mentally alert” are associated with a fourth well-being factor having a variance;
the well-being attributes “calm”, “relaxed”, and “patient” are associated with a fifth well-being factor having a variance
the variance is 48% of the sum of the variances of all well-being factors;
the variance is 24% of the sum of the variances of all well-being factors;
the variance is 12% of the sum of the variances of all well-being factors;
the variance is 9% of the sum of the variances of all well-being factors; and
the variance is 7% of the sum of the variances of all well-being factors.

It was further found that,
within the first well-being factor , the loading of the well-being attribute “not anxious” is 22.2%, the loading of the well-being attribute “not sad” is 21.5%, the loading of the well-being attribute “not restless” is 20.5%, the loading of the well-being attribute “not frustrated” is 20.1%, and the loading of the well-being attribute “not stressed” is 15.7%;
within the second well-being factor , the loading of the well-being attribute “happy” is 19.6%, the loading of the well-being attribute “optimistic” is 19.2%, the loading of the well-being attribute “excited” is 17.2%, the loading of the well-being attribute “satisfied” is 16.3%, the loading of the well-being attribute “motivated” is 13.9%, and the loading of the well-being attribute “invigorated” is 13.8%;
within the third well-being factor , the loading of the well-being attribute “interested” is 51.0%, and the loading of the well-being attribute “not bored” is 49.0%;
within the fourth well-being factor , the loading of the well-being attribute “not fatigued” is 51.0%, and the loading of the well-being attribute “mentally alert” is 49.0%; and
within the fifth well-being factor , the loading of the well-being attribute “calm” is 38.0%, the loading of the well-being attribute “relaxed” is 32.0%, and the loading of the well-being attribute “patient” is 30.0%.

Therefore, in an embodiment of the invention, each well-being attribute has a weight according to the following table:

| well-being attribute | weight | well-being attribute | weight |
|---|---|---|---|
| “not anxious” | 10.65% | “motivated” | 3.34% |
| “not sad” | 10.32% | “invigorated” | 3.31% |
| “not restless” | 9.84% | “interested” | 6.12% |
| “not frustrated” | 9.65% | “not bored” | 5.88% |
| “not stressed” | 7.54% | “not fatigued” | 4.59% |
| “happy” | 4.70% | “mentally alert” | 4.41% |
| “optimistic” | 4.61% | “calm” | 2.66% |
| “excited” | 4.13% | “relaxed” | 2.24% |
| “satisfied” | 3.91% | “patient” | 2.10% |

Preferably, oral care flavour compositions that are not liked by panellists are removed from the list of test oral care flavour compositions to be assessed, as these oral care flavour compositions are unlikely to produce a positive impact on well-being. The degree of liking of the remaining test oral care flavour compositions may still vary according to a pre-defined scale. For example, a vocabulary may be used to define liking, such as “I like this oral care flavour a little”, “this oral care flavour is nice” or “I love this oral care flavour”. Preferably, the intensity of liking may be expressed by moving a slider button over a continuous range of liking, for example from 1 (I dislike it extremely) to 9 (I like it extremely). The slider may be a physical device or virtual, for example on a digital touch screen.

Similarly, sliders may be used advantageously for scoring the well-being attributes mentioned hereinabove. In this case, the continuous scale underlying the motion of the slider preferably goes from a very negative score to a very positive score.

Hence, in an embodiment of the present invention, the strength of the test oral care flavour composition is also assessed by the human subjects, even if care has been taken to equalize the strength of the test oral care flavour compositions.

By performing a well-being assessment on a plurality of generally well-liked oral care flavour compositions at equal strength, it was surprisingly found that the variance of the impact on the well-being was neither correlated with the variance of oral care flavour liking, nor with the variance of the oral care flavour strength as perceived by individual human subjects. This is shown in Example 2. Thus, a positive impact on well-being is independent from liking of the oral care composition. This is a crucial result, which could not be anticipated by the person skilled in the art. An immediate advantage of this is that it makes possible to elucidate hidden functionalities in consumer studies which would otherwise be overlooked.

In an embodiment, the method of the present invention further comprises the steps of:
f) Comparing the oral care flavour compositions having a positive impact on the well-being;
g) Identifying organoleptic attributes common to those oral care flavour compositions; and
h) Creating a new oral care flavour composition with an increased proportion of oral care flavour ingredients with the organoleptic attributes identified in step g).

By “organoleptic attribute” is meant a verbal descriptor describing the odour and taste of a flavour composition. Trigeminal chemesthetic response, such as “cooling”, or “warming”, are also considered as organoleptic attributes. In the context of oral care, the cooling effect of cooling agents is particularly relevant.

Oral care flavour compositions and individual oral care flavour ingredients may be characterized by their organoleptic attributes. Although oral care flavour creation is part science and part artistry, and there is no absolute prescribed definition for organoleptic attributes of oral care flavour compositions and oral care flavour ingredients, nevertheless trained flavourists, realizing that there will be margin for some subjectivity, will be able to assign oral care flavour compositions and ingredients to a general organoleptic attribute.

Organoleptic attributes that are particularly relevant for oral care flavour compositions include anise, peppermint, eucalyptus, spearmint, wintergreen, herbal, citrus, floral, fruity, cooling, warming, spicy, sweet, green, aldehydic, earthy/musky, woody, and animalic.

This selection of organoleptic attributes allows the skilled flavourists to characterize the organoloeptic attribute of all oral care flavour ingredients contained in the flavourist's palette. Nevertheless, for the trained flavourist, reading the contents of this specification as a whole together with their common general knowledge it would not present undue burden to modify part or all of this vocabulary around which there is subjectivity, and such modification would not impact the selection of oral care flavour ingredients useful to impact positively the perception of well-being.

The applicant has now discovered that certain organoleptic attributes impact well-being more positively than others and that a classification of the oral care flavour ingredients according to their impact on the perception of well-being is possible.

In particular, it was found that increasing the proportion of one or more oral care flavour ingredients having an anise organoleptic attribute, and/or the proportion of one or more oral care flavour ingredients having a peppermint organoleptic attribute, and/or the proportion of one or more oral care flavour ingredients having an organoleptic attribute selected from the group consisting of eucalyptus, spearmint, wintergreen, herbal, citrus, floral, fruity, cooling, warming, spicy, and sweet resulted in an enhancement of the well-being of human subjects.

Therefore, in one embodiment of the present invention, the proportion of one or more oral care flavour ingredients having an anise organoleptic attribute, and/or the proportion of one or more oral care flavour ingredients having a peppermint organoleptic attribute, and/or the proportion of one or more oral care flavour ingredients having an organoleptic attribute selected from the group consisting of eucalyptus, spearmint, wintergreen, herbal, citrus, floral, fruity, cooling, warming, spicy, and sweet is increased in step h).

Hence, the present invention teaches the skilled flavourist how to formulate oral care flavour compositions in a manner in which they are likely to arrive quickly at an oral care flavour composition that will induce or be associated with an increase of well-being. The effects are sufficiently pronounced that they can be measured reliably and reproducibly. The oral care flavour compositions made according to the teaching disclosed herein are hedonically pleasant, suitable for a wide range of oral care products, and of sufficient pleasantness/acceptability that they would be appropriate even if they did not possess the added functionality.

The method of the present invention has been found to allow the preparation or optimization of oral care flavour compositions to enhance the well-being of human subjects by:
promoting a reduction in negativity, namely how anxious, sad, restless, frustrated and/or stressed a subject is reporting to feel; and/or
promoting an increase in positivity, namely how happy, optimistic, excited, satisfied, motivated and/or invigorated a subject is reporting to feel; and/or
promoting an increase in interest, namely how more interested and/or less bored a subject is reporting to feel; and/or
promoting a decrease in tiredness, namely how less fatigued and/or more mentally alert a subject is reporting to feel; and/or
promoting an increase in calmness, namely how calm, relaxed and/or patient a subject is reporting to feel.

In the context of the present invention, the term “oral care flavour ingredient” refers to an ingredient that has the function of providing a noticeable and identifiable organoleptic effect to the oral care flavour composition. Oral care flavour ingredients include highly performing ingredients intended for providing an intense olfactive and taste impression, as well as less performing ingredients intended for providing a subtle olfactive and taste impression.

The present invention also provides an oral care flavour composition that allows for enhancing the well-being of human subjects.

The oral care flavour composition of the present invention comprises:
(i) one or more oral care flavour ingredients having an anise organoleptic attribute;
(ii) one or more oral care flavour ingredients having a peppermint organoleptic attribute; and
(iii) at least one oral care flavour ingredient having another organoleptic attribute, wherein said other organoleptic attribute is selected from the group consisting of eucalyptus, spearmint, wintergreen, herbal, citrus, floral, fruity, cooling, warming, spicy, and sweet.

The total amount of oral care flavour ingredients having an anise organoleptic attribute is at least 3.0 wt % of the oral care flavour composition, more preferably at least 5.0 wt %, even more preferably at least 7.0 wt %, and most preferably at least 8.0 wt %. Typically, the amount of oral care flavour ingredients having an anise organoleptic attribute is lower for oral care flavour compositions intended for use in a mouth wash composition, e.g. of at least about 3.0 wt %, and may be higher if the intended use is in another oral care consumer product, e.g. at least 7.0 wt %.

The total amount of oral care flavour ingredients having a peppermint organoleptic attribute is at least 50 wt % of the oral care flavour composition, more preferably at least 55 wt %, and most preferably at least 60 wt %.

Throughout this application, the term “oil” is meant to encompass both fully natural essential oils and extracts, as well as oils derived from natural essential oils and extracts, and modified essential oils and extracts that may comprise additional ingredients; irrespective of the extraction method.

Throughout this application, the term “reconstitution” refers to a mixture of oral care flavour ingredients that provides a similar flavour impression to the corresponding essential oil.

In an embodiment, the one or more oral care flavour ingredients having an anise organoleptic attribute are selected from the group consisting of anethole ((E)-1-methoxy-4-(prop-1-en-1-yl)benzene), anise star oil, anise star oil reconstitutions, tarragon oil, tarragon oil reconstitutions, aniseed oil, aniseed oil reconstitutions, fennel oil, fennel oil reconstitutions, basil oil, basil oil reconstitutions, 1-(cyclopropylmethyl)-4-methoxybenzene (toscanol), and mixtures thereof.

In an embodiment, the one or more oral care flavour ingredients having a peppermint organoleptic attribute are selected from the group consisting of peppermint oil, peppermint oil reconstitutions, L-menthol (L-2-isopropyl-5-methylcyclohexanol), racemic menthol ((D/L)-2-isopropyl-5-methylcyclohexanol), L-isomenthone (L-2-isopropyl-5-methylcyclohexanone), racemic isomenthone ((D/L)-2-isopropyl-5-methylcyclohexanone), menthyl acetate ([(1R,2S,5R)-5-methyl-2-propan-2-ylcyclohexyl]acetate), menthofuran (3,5-dimethyl-4,5,6,7-tetrahydro-1-benzofuran), 2-butan-2-ylcyclohexan-1-one (Freskomenthe), and mixtures thereof.

In an embodiment, the oral care flavour composition of the present invention comprises (iii.1) one or more oral care flavour ingredients having an eucalyptus organoleptic attribute, wherein the total amount of oral care flavour ingredients having an eucalyptus organoleptic attribute is at least 20 wt % of the oral care flavour composition, more preferably at least 25 wt %, and most preferably at least 30 wt %.

In an embodiment, the one or more oral care flavour ingredients having an eucalyptus organoleptic attribute is selected from the group consisting of eucalyptus oil, eucalyptus oil reconstitutions, eucalyptol ((1S,4S)-1,3,3-trimethyl-2-oxabicyclo[2.2.2]octane), and mixtures thereof.

In an embodiment, the oral care flavour composition of the present invention comprises (iii.2) one or more oral care flavour ingredients having a spearmint organoleptic attribute, wherein the total amount of oral care flavour ingredients having a spearmint organoleptic attribute is at least 20 wt % of the oral care flavour composition, more preferably at least 25 wt %, and most preferably at least 30 wt %.

In an embodiment, the one or more oral care flavour ingredients having a spearmint organoleptic attribute is selected from the group consisting of spearmint oil, spearmint oil reconstitutions, trans-carveol ((1R,5S)-2-methyl-5-prop-1-en-2-ylcyclohex-2-en-1-ol), cis-carveol ((1R,5R)-2-methyl-5-prop-1-en-2-ylcyclohex-2-en-1-ol), L-carvone ((5R)-2-methyl-5-prop-1-en-2-ylcyclohex-2-en-1-one), and mixtures thereof.

In an embodiment, the oral care flavour composition of the present invention comprises (iii.3) one or more oral care flavour ingredients having a wintergreen organoleptic attribute, wherein the total amount of oral care flavour ingredients having a wintergreen organoleptic attribute is at least 20 wt % of the oral care flavour composition, more preferably at least 25 wt %, and most preferably at least 30 wt %.

In an embodiment, the one or more oral care flavour ingredients having a wintergreen organoleptic attribute is selected from the group consisting of wintergreen oil, wintergreen oil reconsititutions, ethyl salicylate (ethyl 2-hydroxybenzoate), methyl salicylate (methyl 2-hydroxybenzoate), and mixtures thereof.

In an embodiment, the oral care flavour composition of the present invention comprises (iii.4) one or more oral care flavour ingredients having a herbal organoleptic attribute, wherein the total amount of oral care flavour ingredients having a herbal organoleptic attribute is at least 10 wt % of the oral care flavour composition, more preferably at least 12 wt %, and most preferably at least 15 wt %.

In general, the term “herbal organoleptic attribute” is meant to encompass all flavour directions derived from herbs, other than those listed separately as specific organoleptic attributes used in the present invention. This includes, among others, chamomile, rosemary, sage, thyme, lavender, and juniper.

In an embodiment, the one or more oral care flavour ingredients having a herbal organoleptic attribute is selected from the group consisting of camomile oil, camomile oil reconsititutions, juniper berry oil, juniper berry oil reconstitutions, lavandin oil, lavandin oil reconstitutions, lavender oil, lavender oil reconstitutions, rosemary oil, rosemary oil reconstitutions, sage oil, sage oil reconstitutions, thyme oil, thyme oil reconsititutions, thymol, and mixtures thereof.

In an embodiment, the oral care flavour composition of the present invention comprises (iii.5) one or more oral care flavour ingredients having a citrus organoleptic attribute, wherein the total amount of oral care flavour ingredients having a citrus organoleptic attribute is at least 20 wt % of the oral care flavour composition, more preferably at least 25 wt %, and most preferably at least 30 wt %.

In an embodiment, the one or more oral care flavour ingredients having a citrus organoleptic attribute is selected from the group consisting of bergamot oil, bergamot oil reconstitutions, citral ((E)-3,7-dimethylocta-2,6-dienal), grapefruit oil, grapefruit oil reconstitutions, lemongrass oil, lemongrass oil reconstitutions, lemon oil, lemon oil reconstitutions, D-limonene ((4R)-1-methyl-4-prop-1-en-2-ylcyclohex-1-ene), L-limonene ((4S)-1-methyl-4-prop-1-en-2-ylcyclohex-1-ene), lime oil, lime oil reconstitutions, mandarin oil, mandarin oil reconstitutions, orange oil, orange oil reconstitutions, orange bitter oil, orange bitter oil reconstitutions, orange blood oil, orange blood oil reconstitutions, tangerine oil, tangerine oil reconsititutions, and mixtures thereof.

In an embodiment, the oral care flavour composition of the present invention comprises (iii.6) one or more oral care flavour ingredients having a floral organoleptic attribute, wherein the total amount of oral care flavour ingredients having a floral organoleptic attribute is at least 5.0 wt % of the oral care flavour composition, more preferably at least 8.0 wt %, and most preferably at least 12.0 wt %.

In an embodiment, the one or more oral care flavour ingredients having a floral organoleptic attribute is selected from the group consisting of citronellol (3,7-dimethyloct-6-en-1-ol), geranium oil, geranium oil reconstitutions, geranyl acetate ((E)-3,7-dimethylocta-2,6-dien-1-yl acetate), geraniol ((E)-3,7-dimethylocta-2,6-dien-1-ol), hydroxycitronellal (7-hydroxy-3,7-dimethyloctanal), ionone alpha ((E)-4-(2,6,6-trimethyl-1-cyclohex-2-enyl)but-3-en-2-one), ionone beta ((E)-4-(2,6,6-trimethylcyclohex-1-en-1-yl)but-3-en-2-one), jasmin oil, jasmin oil reconstitutions, linalool (3,7-dimethylocta-1,6-dien-3-ol), phenyl ethyl alcohol (2-phenylethanol), a mixture of 3,7-dimethyloct-6-en-1-ol and 3,7-dimethyloct-7-en-1-ol (rhodinol), tetrahydro geraniol, rose oil, rose oil reconstitutions, terpineol alpha(2-(4-methyl-1-cyclohex-3-enyl)propan-2-ol), ylang ylang oil, ylang ylang oil reconstitutions, and mixtures thereof.

In an embodiment, the oral care flavour composition of the present invention comprises (iii.7) one or more oral care flavour ingredients having a fruity organoleptic attribute, wherein the total amount of oral care flavour ingredients having a fruity organoleptic attribute is at least 8.0 wt % of the oral care flavour composition, more preferably at least 10.0 wt %, and most preferably at least 12.0 wt %.

In an embodiment, the one or more oral care flavour ingredients having a fruity organoleptic attribute is selected from the group consisting of cassyrane (5-tert-butyl-2-methyl-5-propyl-2H-furan), cis-3-hexenyl acetate ((Z)-hex-3-enyl acetate), damascenone ((E)-1-(2,6,6-trimethylcyclohexa-1,3-dien-1-yl)but-2-en-1-one), damascone beta ((E)-1-(2,6,6-trimethyl-1-cyclohexenyl)but-2-en-1-one), decalactone gamma (5-hexyloxolan-2-one), dimethyl sulphide ((methylsulfanyl)methane), dodecalactone gamma (5-octyloxolan-2-one), ethyl 2-methyl butyrate (ethyl 2-methylbutanoate), ethyl acetate, ethyl butyrate (ethyl butanoate), ethyl heptanoate, ethyl hexanoate, ethyl iso-butyrate (ethyl 2-methylpropionate), ethyl lactate (ethyl 2-hydroxypropanoate), hexyl acetate, iso-amyl acetate (3-methylbutyl acetate), iso-amyl butyrate (3-methylbutyl butanoate), iso-amyl propionate (3-methylbutyl propionate), raspberry ketone (4-(4-hydroxyphenyl)butan-2-one), trans-2-hexenal, and mixtures thereof.

In an embodiment, the oral care flavour composition of the present invention comprises (iii.8) one or more oral care flavour ingredients having a cooling organoleptic attribute, wherein the total amount of oral care flavour ingredients having a cooling organoleptic attribute is at least 1.0 wt % of the oral care flavour composition, more preferably at least 1.5 wt %, and most preferably at least 2.0 wt %. The person skilled in the art will be well aware that intensity of oral care flavour ingredients having a cooling organoleptic attribute may vary, depending on the ingredient, and that, therefore, also the required amount may vary. In particular, high intensity coolants, such as e.g. 2-(p-tolyloxy)-N-(1H-pyrazol-5-yl)-N-((thiophen-2-yl)methyl)acetamide (Freezestorm), may be used in lower amounts than those mentioned before.

In an embodiment, the one or more oral care flavour ingredients having a cooling organoleptic attribute is selected from the group consisting of N-(4-cyanomethylphenyl) p-menthane carboxamide (Evercool 180), 2-isopropyl-5-methyl-N-(2-(pyridin-2-yl)ethyl)cyclohexane carboxamide (Evercool 190), 3-(5-methyl-2-propan-2-ylcyclohexyl)oxypropane-1,2-diol (Coolact P), (1R,2R,5S)-5-methyl-2-prop-1-en-2-ylcyclohexan-1-ol (Coolact 10), ethyl 2-[[(1R,2S,5R)-5-methyl-2-propan-2-ylcyclohexanecarbonyl]amino]acetate (WS-5), (1R,2S,5R)-N-(4-methoxyphenyl)-5-methyl-2-(1-methylethyl)cyclohexanecarboxamide (WS-12), N-ethyl-5-methyl-2-propan-2-ylcyclohexane-1-carboxamide (WS-3), N,2,3-trimethyl-2-propan-2-ylbutanamide (WS-23), (2E)-3-(1,3-benzodioxol-5-yl)-N,N-diphenylacrylamide (ICool MC6), 2-(p-tolyloxy)-N-(1H-pyrazol-5-yl)-N-((thiophen-2-yl)methyl)acetamide (Freezestorm), [(1R,2S,5R)-5-methyl-2-propan-2-ylcyclohexyl]2-hydroxypropanoate (Frescolat ML), 2-isopropyl-5-methylcyclohexyloxycarbonyloxy-2-hydroxypropane (Frescolat MGC), 2-hydroxypropyl (5-methyl-2-propan-2-ylcyclohexyl) carbonate (Frescolat MPC), 9-methyl-6-(1-methylethyl)-1,4-dioxaspiro-[4,5]decan-2-methanol (Frescolat MGA), menthyl lactate, mono-menthyl succinate, menthyl methyl ether, 1-methyl-3-hydroxybutyrate, 2-{[5-methyl-2-(propan-2-yl)cyclohexyl]oxy}ethanol (Coolact 5), 2-(2-hydroxypropan-2-yl)-5-methylcyclohexan-1-ol (Coolact 38D), and mixtures thereof.

In an embodiment, the oral care flavour composition of the present invention comprises (iii.9) one or more oral care flavour ingredients having a warming organoleptic attribute, wherein the total amount of oral care flavour ingredients having a warming organoleptic attribute is at least 0.10 wt % of the oral care flavour composition, more preferably at least 0.15 wt %, and most preferably at least 0.20 wt %.

In an embodiment, the one or more oral care flavour ingredients having a warming organoleptic attribute is selected from the group consisting of capsaicine, capsicum oleoresin, gingerol, nonivamide, piperine, vanillyl butyl ether, and mixtures thereof.

In an embodiment, the oral care flavour composition of the present invention comprises (iii.10) one or more oral care flavour ingredients having a spicy organoleptic attribute, wherein the total amount of oral care flavour ingredients having a spicy organoleptic attribute is at least 10 wt % of the oral care flavour composition, more preferably at least 15 wt %, and most preferably at least 20 wt %.

In an embodiment, the one or more oral care flavour ingredients having a spicy organoleptic attribute is selected from the group consisting of cassia oil, cassia oil reconstitutions, cinnamic aldehyde, clove bud oil, clove bud oil reconstitutions, clove leaf oil, clove leaf oil reconstitutions, eugenol, ginger oil, ginger oil reconstitutions, ginger oleoresin, black pepper oil, black pepper oil reconstitutions, cardamom oil, cardamom oil reconstitutions, nutmeg oil, nutmeg oil reconstitutions, cinnamon leaf oil, cinnamon leaf oil reconstitutions, cinnamon bark oil, cinnamon bark oil reconstitutions, safraleine (2,3,3-trimethyl-2,3-dihydro-1H-inden-1-one), and mixtures thereof.

In an embodiment, the oral care flavour composition of the present invention comprises (iii.11) one or more oral care flavour ingredients having a sweet organoleptic attribute, wherein the total amount of oral care flavour ingredients having a sweet organoleptic attribute is at least 10 wt % of the oral care flavour composition, more preferably at least 15 wt %, and most preferably at least 20 wt %.

In an embodiment, the one or more oral care flavour ingredients having a sweet organoleptic attribute is selected from the group consisting of acetyl methyl carbinol (3-hydroxypentan-2-one), benzaldehyde, cinnamic alcohol, decalactone delta (6-pentyltetrahydro-2H-pyran-2-one), ethyl maltol (2-ethyl-3-hydroxy-4H-pyran-4-one), fenugreek oil, fenugreek oil reconstitutions, furaneol (4-hydroxy-2,5-dimethylfuran-3-one), hexalactone gamma (5-ethyloxolan-2-one), heliotropin (benzo[d][1,3]dioxole-5-carbaldehyde), isobutavan (4-formyl-2-methoxyphenyl 2-methylpropanoate), nonalactone gamma (5-pentyloxolan-2-one), octalactone gamma (5-butyloxolan-2-one), p-methyl acetophenone (1-(4-methylphenyl)ethanone), maltol (3-hydroxy-2-methyl-4H-pyran-4-one), myrrh oil, myrrh oil reconstitutions, vanilla oil, vanilla oil reconstitutions, vanillin (4-hydroxy-3-methoxybenzaldehyde), and mixtures thereof.

The oral care flavour composition of the present invention may comprise further oral care flavour ingredients, having other organoleptic attributes. For instance, the oral care flavour composition may comprise one or more of the following:
(iv.1) one or more oral care flavour ingredients having an aldehydic organoleptic attribute; and/or
(iv.2) one or more oral care flavour ingredients having an animalic organoleptic attribute; and/or
(iv.3) one or more oral care flavour ingredients having a green leaf organoleptic attribute; and/or
(iv.4) one or more oral care flavour ingredients having an earthy/musky organoleptic attribute.

In an embodiment, the total amount of oral care flavour ingredients having an aldehydic organoleptic attribute is at least 0.10 wt % of the oral care flavour composition, more preferably at least 0.15 wt %, and most preferably at least 0.20 wt %.

In an embodiment, the total amount of oral care flavour ingredients having an animalic organoleptic attribute is at least 1.0 wt % of the oral care flavour composition, more preferably at least 1.5 wt %, and most preferably at least 2.0 wt %.

In an embodiment, the total amount of oral care flavour ingredients having a green leaf organoleptic attribute is at least 0.05 wt % of the oral care flavour composition, more preferably at least 0.10 wt %, and most preferably at least 0.20 wt %.

In an embodiment, wherein the total amount of oral care flavour ingredients having a earthy organoleptic attribute is at least 0.10 wt % of the oral care flavour composition, more preferably at least 0.15 wt %, and most preferably at least 0.20 wt %.

The oral care flavour compositions of the present invention may be presented in the form of free oils, or they may be encapsulated. Several encapsulating media are known in the art for encapsulating oral care

flavour compositions. Particular encapsulating media include microcapsules formed of natural or modified natural polymers, such as polysaccharides or proteins.

The oral care flavour compositions of the present invention may be used to impart desirable odour impressions on all manner of oral care consumer products.

Oral care consumer products include toothpaste, dental cream, gel or tooth powder, odontic, denture tablets, denture cleaners, denture fixatives, mouth washes, including plaque removing liquids, typically comprise a water/alcohol solution, humectant, sweetener, foaming agent, colorant, and optionally enzymes; pre- or post-brushing rinse formulations, chewing gum, lozenges, and candy. Toothpastes and tooth gels typically include abrasive polishing materials, foaming agents, flavouring agents, humectants, binders, thickeners, sweetening agents, whitening/bleaching/stain removing agents, water, and optionally enzymes.

Oral care consumer products comprise formulated mixtures of various functional ingredients, such as surfactants, emulsifiers, polymers, fillers and solvents. These formulated mixtures are usually referred to as “bases”.

The methods of the present invention may be facilitated by computer means.

The computer means may comprise suitable hardware and software components commonly known and used in the art.

The computer means may comprise a database of oral care flavour ingredients or oral care flavour compositions in computer readable form. For each oral care flavour ingredient or oral care flavour composition, the database may contain at least one defining parameter, property or function of that ingredient or composition. In particular, for each oral care flavour ingredient or composition, there may be assigned a well-being score defined according to the methods described above.

The computer means may comprise data-processing means configured and operable to allow a formulator to select oral care flavour ingredients from the database, and to calculate a well-being score for a oral care flavour composition containing the selected oral care flavour ingredients.

Hereinafter, the present invention will be described in further detail with reference to examples. It is to be understood, however, that these examples are for illustrative purposes only and are not to be construed to limit the scope of the present invention.

### EXAMPLE 1

### Development of the Well-Being Metric (WB-18)

A. Identification of well-being attributes

In a preliminary study, a broad selection of verbal well-being attributes was considered and those which were shown to be sensitive to the action of odour were retained. The most relevant of these attributes were those which showed significant differences between control, malodours and/or perfume conditions and, importantly, between pleasant perfumes. This led to a subset of 18 attributes, which showed the highest sensitivity to perfume, according to principal component analysis. The thus identified WB-18 attributes are listed in Table 1 below.

Each item was assessed by panellists moving a virtual slider on a touch screen. 138 panellists across four different studies completed the assessment.

Panellists first completed two baseline measures of the WB-18 attributes in the absence of and before exposure to a test perfume composition, with the second baseline measure being completed immediately after the first one. Factor analysis (see above) was applied to the averaged baseline data to determine five well-being factors and the related variances, as well as the loadings for each well-being attribute within the five well-being factors (see Table 1). Factor loadings are based on a principal axis factoring with oblimin rotation because of the possible correlations between some of the well-being attributes.

TABLE 1
Well-being attributes, factors and factor loadings
obtained from principal component analysis

| | Factor 1 | Factor 2 | Factor 3 | Factor 4 | Factor 5 |
|---|---|---|---|---|---|
| | (vari- | (vari- | (vari- | (vari- | (vari- |
| | ance = | ance = | ance = | ance = | ance = |
| Attribute | 0.48) | 0.24) | 0.12) | 0.09) | 0.07) |
| Not anxious | 0.222 | | | | |
| Not sad | 0.215 | | | | |
| Not restless | 0.205 | | | | |
| Not frustrated | 0.201 | | | | |
| Not stressed | 0.157 | | | | |
| Happy | | 0.196 | | | |
| Optimistic | | 0.192 | | | |
| Excited | | 0.172 | | | |
| Satisfied | | 0.163 | | | |
| Motivated | | 0.139 | | | |
| Invigorated | | 0.138 | | | |
| Interested | | | 0.510 | | |
| Not bored | | | 0.490 | | |
| Not fatigued | | | | 0.510 | |
| Mentally alert | | | | 0.490 | |
| Calm | | | | | 0.380 |
| Relaxed | | | | | 0.320 |
| Patient | | | | | 0.300 |

Based on the above analysis, the weighting for each well-being attribute was determined as shown in Table 2:

TABLE 2
Weighting of the well-being attributes

| well-being attribute | weight | well-being attribute | weight |
|---|---|---|---|
| “not anxious” | 10.65% | “motivated” | 3.34% |
| “not sad” | 10.32% | “invigorated” | 3.31% |
| “not restless” | 9.84% | “interested” | 6.12% |
| “not frustrated” | 9.65% | “not bored” | 5.88% |
| “not stressed” | 7.54% | “not fatigued” | 4.59% |
| “happy” | 4.70% | “mentally alert” | 4.41% |
| “optimistic” | 4.61% | “calm” | 2.66% |
| “excited” | 4.13% | “relaxed” | 2.24% |
| “satisfied” | 3.91% | “patient” | 2.10% |

B. Methodology protocol for well-being study

539 subjects between the ages of 17 and 61 (264 male and 275 female) were recruited for this study. Subjects were native English speakers with normal or corrected vision, in good oral health, and recruited from the general public in the UK. All subjects indicated that they had no known deficits in their sense of smell, olfactory sensitivities or neurological diseases. Test oral care flavour compositions were incorporated in a standard silica opacified toothpaste at a level of 1 wt.-%, together with sweetener sodium saccharin dosed at 0.25 wt.-%, based on the total weight of the toothpaste. Each participant received six different samples with test oral care flavour compositions, with instructions to smell and score online one sample per day at approximately the same time of their choosing.

Each day, the participants first scored the WB-18 attributes before brushing their teeth and in the absence of a test oral care flavour composition.

Participants then brushed their teeth with the toothpaste containing the test oral care flavour composition and scored the WB-18 attributes within 5 min after the action of teeth brushing had been completed.

The well-being attributes were randomised for each participant for each assessment.

The particpants also provided a score related on how strongly they liked or disliked each flavour, providing a liking rating on a scale of 1 (I dislike it extremely) to 9 (I like it extremely).

| I | I | I | I | I neither | I | I | I | I |
|---|---|---|---|---|---|---|---|---|
| dislike it | dislike it | dislike it | dislike it | like nor | like it | like it | like it | like it |
| extremely | very much | moderately | slightly | dislike it | slightly | moderately | very much | extremely |
| 1 | 2 | 3 | 4 | 5 | 6 | 7 | 8 | 9 |

In addition, participants provided a strength rating on a scale of 1 (extremely weak) to 9 (extremely strong).

| Extremely | Very | Quite | Slightly | Neither weak | Slightly | Quite | Very | Extremely |
|---|---|---|---|---|---|---|---|---|
| weak | weak | weak | weak | nor strong | strong | strong | strong | strong |
| 1 | 2 | 3 | 4 | 5 | 6 | 7 | 8 | 9 |

C. Test oral care flavour compositions

The following 11 test oral care flavour compositions were prepared and tested in the present study (Table 3):

TABLE 3
Test oral care flavour compositions

| | Organoleptic | Formulation (% by wt) | | | | | | | | | | |
|---|---|---|---|---|---|---|---|---|---|---|---|---|
| Ingredient | Attribute | 1 | 2 | 3 | 4 | 5 | 6 | 7 | 8 | 9 | 10 | 11 |
| Aldehyde C9 (Nonanal) | ALDEHYDIC | 0.17 | | | | | | | | | | |
| 2 methyl butyric acid | ANIMALIC | 2.16 | | | | | | | | | | |
| Anethole | ANISE | 8.00 | 8.00 | 8.00 | 8.00 | 7.68 | 7.58 | 8.00 | 8.00 | 8.00 | 8.00 | 8.00 |
| Anise Star oil | ANISE | | | | | | | | | | | 2.00 |
| Basil Egypt orpur | ANISE | | | | | | | | | | | 0.50 |
| Fennel Sweet orpur | ANISE | | | | | | | | | | | 2.00 |
| Citral | CITRUS | | | | | | | | | | 1.00 | |
| Lemon oils | CITRUS | | | | | | | | | | 18.00 | |
| Lime oils | CITRUS | | | | | | | | | | 18.00 | |
| Evercool 180 | COOLING | | | | | | 1.50 | | | | | |
| Evercool 190 | COOLING | | | | | 4.00 | | | | | | |
| Terpinenol 4 | EARTHY/MUSKY | 0.22 | | | | | | | | | | |
| Cyclopentadecanolide | EARTHY/MUSKY | 0.03 | | | | | | | | | | |
| Eucalyptol (1-8 cineol) | EUCALYPTUS | | 32.00 | | | | | | | | | |
| Eucalyptus oil | EUCALYPTUS | | 5.00 | | | | | | | | | |
| Linalol | FLORAL | 1.40 | | | | | | | 0.40 | | | |
| Terpineol alpha | FLORAL | 0.87 | | | | | | | | | | |
| Hydroxycitronellal | FLORAL | | | | | | | | 0.05 | | | |
| Citronellol pure | FLORAL | | | | | | | | 0.54 | | | |
| Geraniol pure | FLORAL | 0.11 | | | | | | | 6.90 | | | |
| Geranium oils | FLORAL | | | | | | | | 0.40 | | | |
| Geranyl acetate | FLORAL | | | | | | | | 0.80 | | | |
| Phenyl ethyl alcohol | FLORAL | | | | | | | | 3.05 | | | |
| Rhodinol | FLORAL | | | | | | | | 0.80 | | | |
| Rose oils | FLORAL | | | | | | | | 0.02 | | | |
| Rose oil reconstitutions | FLORAL | 0.87 | | | | | | | 0.02 | | | |
| Tetrahydro geraniol | FLORAL | | | | | | | | 0.06 | | | |
| Ionone alpha | FLORAL | 0.35 | | | | | | | 0.13 | | | |
| Ionone beta | FLORAL | 1.57 | | | | | | | 0.03 | | | |
| cis 3 hexenyl acetate | FRUITY | 0.70 | | | | | | | | | | |
| Ethyl 2 methyl butyrate | FRUITY | 2.18 | | | | | | | | | | |
| trans 2 hexenal | FRUITY | 0.44 | | | | | | | | | | |
| Damascenone | FRUITY | 0.09 | | | | | | | 0.10 | | | |
| Decalactone Gamma | FRUITY | 0.44 | | | | | | | | | | |
| Ethyl heptanoate | FRUITY | | | 0.43 | | | | | | | | |
| Ethyl hexanoate | FRUITY | 0.61 | | | | | | | | | | |
| Raspberry ketone | FRUITY | 6.98 | | | | | | | | | | |
| Ethyl Iso Butyrate | FRUITY | 2.71 | | | | | | | | | | |
| Dimethyl sulphide | FRUITY | 0.31 | | | | | | | | | | |
| Aldehyde C6(Elexanal) | GREEN | 0.22 | | | | | | | | | | |
| Phenyl acetic aldehyde 50% | GREEN | | | | | | | | 0.02 | | | |
| PEA | | | | | | | | | | | | |
| Phenylyacetaldehyde | GREEN | | | | | | | | 0.04 | | | |
| dimethyl acetal | | | | | | | | | | | | |
| Chamomile England | HERBAL | | | | | | | | | | | 0.30 |
| Rosemary oil | HERBAL | | | | | | | | | | | 8.00 |
| Sage oil | HERBAL | | | | | | | | | | | 8.00 |
| Thyme White oil | HERBAL | | | | | | | | | | | 0.50 |
| Thymol | HERBAL | | | | | | | | | | | 0.50 |
| Propylene glycol | ODOURLESS | 5.34 | | 17.00 | 5.00 | | 0.27 | | 23.64 | 17.00 | | 15.20 |
| L-menthol | PEPPERMINT | 45.00 | 45.00 | 45.00 | 45.00 | 43.20 | 44.33 | 45.00 | 45.00 | 45.00 | 45.00 | 45.00 |
| Peppermint oils | PEPPERMINT | | | | | 21.12 | 21.68 | | | | | |
| Peppermint oil | PEPPERMINT | 11.54 | 10.00 | 10.00 | 10.00 | 24.00 | 24.64 | 10.00 | 10.00 | 10.00 | 10.00 | 10.00 |
| reconstitutions | | | | | | | | | | | | |
| L- Carvone | SPEARMINT | | | | 10.00 | | | | | | | |
| Spearmint oils | SPEARMINT | | | | 17.00 | | | | | | | |
| Spearmint oil | SPEARMINT | | | | 5.00 | | | | | | | |
| recontitutions | | | | | | | | | | | | |
| Cassia oil | SPICY | | | | | | | | | 5.00 | | |
| Cinnamic aldehyde | SPICY | | | | | | | | | 5.00 | | |
| Clove bud oil | SPICY | | | | | | | | | 5.00 | | |
| Eugenol | SPICY | | | | | | | | | 5.00 | | |
| Hexalactone gamma | SWEET | 0.35 | | | | | | | | | | |
| Nonalactone gamma | SWEET | | | 12.23 | | | | | | | | |
| Octalactone Gamma | SWEET | | | 2.72 | | | | | | | | |
| Furaneol (10%) | SWEET | 7.14 | | | | | | | | | | |
| Vanilla absolute | SWEET | | | 0.10 | | | | | | | | |
| Vanillin | SWEET | | | 4.52 | | | | | | | | |
| Capsicum oleoresin | WARMING | 0.20 | | | | | | | | | | |
| Methyl Salicylate | WINTERGREEN | | | | | | | 37.00 | | | | |

D. Measurement of the impact of the test oral care flavour compositions on well-being

12 test toothpastes were prepared, one each with the 11 test oral care flavour compositions from section C (Table 3) and one of the toothpastes remaining unflavoured. The test toothpastes were submitted to the participants of the study for well-being assessment according to the method described in Section B.

The participants assessed the WB-18 attributes providing a first individual well-being score (=base) well-being score) for each well-being attribute before brushing their teeth and in the absence of a test oral care flavour composition (w/o) and a second individual well-being score immediately after having brushed their teeth in the presence of each test oral care flavour composition (w). The corresponding well-being scores were calculated by multiplying the individual score of each well-being attribute by the weighting as determined in Section A (see Table 2) and summing up the individual scores.

Finally, the well-being enhancement score was calculated by subtracting the base well-being score (i.e. the sum of the first individual well-being scores multiplied by their weightings) from the well-being score with the test oral care flavour composition (i.e. the sum of the second individual well-being scores multiplied by their weightings). Average results over all participants are shown in Table 4.

TABLE 4
Averaged well-being scores before and after teeth brushing with
the test toothpastes, and well-being enhancement in percent

| Oral care flavour | Before teeth | After teeth | Well-being |
|---|---|---|---|
| composition | brushing | brushing | enhancement score |
| 1 | 7.17 | 7.48 | 4.1% |
| 2 | 7.38 | 7.67 | 3.8% |
| 3 | 7.37 | 7.66 | 3.8% |
| 4 | 7.17 | 7.43 | 3.5% |
| 5 | 7.42 | 7.63 | 2.8% |
| 6 | 7.48 | 7.62 | 1.8% |
| 7 | 7.2 | 7.33 | 1.8% |
| 8 | 7.44 | 7.54 | 1.3% |
| 9 | 7.55 | 7.64 | 1.2% |
| 10 | 7.31 | 7.38 | 0.9% |
| 11 | 7.57 | 7.63 | 0.8% |
| Unflavoured | 7.57 | 7.41 | −2.2% |

### EXAMPLE 2

### Lack of Correlation Between Well-Being Perception and Liking/Strength

In order to confirm that the impact on well-being is not coupled to liking and/or strength of the test oral care flavour compositions, a principal component analysis was performed on the panellists' individual ratings of liking and strength (Example 1, Section B), and well-being score (Example 1, Section D). The corresponding correlation coefficients are reported in Table 5:

TABLE 5
Correlation matrix relating oral care flavour liking,
oral care flavour strength and well-being score

| | Overall | Flavour | | |
|---|---|---|---|---|
| | Liking | Liking | Strength | Well-being |
| Overall Liking | 1 | | | |
| Flavour Liking | 0.9 | 1 | | |
| Strength | 0.7 | 0.7 | 1 | |
| Well-being | 0.2 | 0.2 | 0.1 | 1 |

As apparent from Table 5, the well-being score is neither correlated to the variations of the oral care flavour liking from one panellist to another nor to the variation of oral care flavour strength perception from one panellist to another.

1. A method of assessing an impact of a test oral care flavour composition on the well-being of human subjects, by performing the steps of:
a) Having one or more human subjects assess their well-being before brushing their teeth and in the absence of the test oral care flavour composition by scoring a plurality of well-being attributes;
i. wherein the well-being attributes include “not anxious”, “not sad”, “not restless”, “not frustrated”, “not stressed”, “happy”, “optimistic”, “excited”, “satisfied”, “motivated”, “invigorated”, “interested”, “not bored”, “not fatigued”, “mentally alert”, “calm”, “relaxed”, and “patient”; and
ii. wherein each well-being attribute has a corresponding weight;
b) Calculating a base well-being score before teeth brushing and in the absence of the test oral care flavour composition by multiplying the score of each well-being attribute by the corresponding weight to form a product and summing these products over all well-being attributes;
c) Repeating steps a) and b) after teeth brushing with a toothpast comprising the test oral care flavour composition in order to determine a well-being score with the test oral care flavour composition; and
d) Determining the impact of the test oral care flavour composition on the well-being by subtracting the base well-being score from the well-being score with the test oral care flavour composition.

2. The method of claim 1, wherein the weights of the well-being attributes are as follows:

| well-being attribute | weight |
|---|---|
| “not anxious” | 10.65% |
| “not sad” | 10.32% |
| “not restless” | 9.84% |
| “not frustrated” | 9.65% |
| “not stressed” | 7.54% |
| “happy” | 4.70% |
| “optimistic” | 4.61% |
| “excited” | 4.13% |
| “satisfied” | 3.91% |
| “motivated” | 3.34% |
| “invigorated” | 3.31% |
| “interested” | 6.12% |
| “not bored” | 5.88% |
| “not fatigued” | 4.59% |
| “mentally alert” | 4.41% |
| “calm” | 2.66% |
| “relaxed” | 2.24% |
| “patient” | 2.10% |

3. A method of preparing or optimizing an oral care flavour composition for enhancing the well-being of a human subject, the method comprising the steps of:
a-d) Determining the impact of one or more oral care flavour compositions on the well-being of human subjects according to the method of claim 1; and
e) Selecting an oral care flavour composition with a positive impact on the well-being.

4. The method of claim 3, further comprising the steps of:
f) comparing the oral care flavour ingredients present in those oral care flavour compositions having a positive impact on the well-being;
g) identifying organoleptic attributes common to those oral care flavour compositions; and
h) creating a new oral care flavour composition with an increased proportion of oral care flavour ingredients with the organoleptic attributes identified in step g).

5. The method according to claim 4, wherein in step h), the proportion of one or more oral care flavour ingredients having an anise organoleptic attribute is increased, and/or the proportion of one or more oral care flavour ingredients having a peppermint organoleptic attribute is increased, and/or the proportion of one or more oral care flavour ingredients having an organoleptic attribute selected from the group consisting of eucalyptus, spearmint, wintergreen, herbal, citrus, floral, fruity, cooling, warming, spicy, and sweet is increased.

6. An oral care flavour composition comprising:
(i) one or more oral care flavour ingredients having an anise organoleptic attribute, wherein the total amount of oral care flavour ingredients having an anise organoleptic attribute is at least 3.0 wt % of the oral care flavour composition;
(ii) one or more oral care flavour ingredients having a peppermint organoleptic attribute, wherein the total amount of oral care flavour ingredients having a peppermint organoleptic attribute is at least 50 wt % of the oral care flavour composition:
(iii) at least one oral care flavour ingredient having another organoleptic attribute, wherein said other organoleptic attribute is selected from the group consisting of eucalyptus, spearmint, wintergreen, herbal, citrus, floral, fruity, cooling, warming, spicy, and sweet.

7. The oral care flavour composition according to claim 6, wherein the one or more oral care flavour ingredients having an anise organoleptic attribute are selected from the group consisting of anethole, anise star oil, anise star oil reconstitutions, tarragon oil, tarragon oil reconstitutions, aniseed oil, aniseed oil reconstitutions, fennel oil, fennel oil reconstitutions, basil oil, basil oil reconstitutions, (1-(cyclopropylmethyl)-4-methoxybenzene), and mixtures thereof.

8. The oral care flavour composition according to claim 6, wherein the one or more oral care flavour ingredients having a peppermint organoleptic attribute are selected from the group consisting of peppermint oil, peppermint oil reconstitutions, L-menthol, racemic menthol, L-isomenthone, racemic isomenthone, menthyl acetate, menthofuran, 2-butan-2-ylcyclohexan-1-one, and mixtures thereof.

9. The oral care flavour composition according to claim 6, comprising at least one of (iii.1) to (iii.11) in the following amount:
(iii.1) one or more oral care flavour ingredients having an eucalyptus organoleptic attribute, wherein the total amount of oral care flavour ingredients having an eucalyptus organoleptic attribute is at least 20 wt % of the oral care flavour composition, and/or
(iii.2) one or more oral care flavour ingredients having a spearmint organoleptic attribute, wherein the total amount of oral care flavour ingredients having a spearmint organoleptic attribute is at least 20 wt % of the oral care flavour composition, and/or
(iii.3) one or more oral care flavour ingredients having a wintergreen organoleptic attribute, wherein the total amount of oral care flavour ingredients having a wintergreen organoleptic attribute is at least 20 wt % of the oral care flavour composition, and/or
(iii.4) one or more oral care flavour ingredients having a herbal organoleptic attribute, wherein the total amount of oral care flavour ingredients having a herbal organoleptic attribute is at least 10 wt % of the oral care flavour composition, and/or
(iii.5) one or more oral care flavour ingredients having a citrus organoleptic attribute, wherein the total amount of oral care flavour ingredients having a citrus organoleptic attribute is at least 20 wt % of the oral care flavour composition, and/or
(iii.6) one or more oral care flavour ingredients having a floral organoleptic attribute, wherein the total amount of oral care flavour ingredients having a floral organoleptic attribute is at least 5.0 wt % of the oral care flavour composition, and/or
(iii.7) one or more oral care flavour ingredients having a fruity organoleptic attribute, wherein the total amount of oral care flavour ingredients having a fruity organoleptic attribute is at least 8.0 wt % of the oral care flavour composition, and/or
(iii.8) one or more oral care flavour ingredients having a cooling organoleptic attribute, wherein the total amount of oral care flavour ingredients having a cooling organoleptic attribute is at least 1.0 wt % of the oral care flavour composition, and/or
(iii.9) one or more oral care flavour ingredients having a warming organoleptic attribute, wherein the total amount of oral care flavour ingredients having a warming organoleptic attribute is at least 0.10 wt % of the oral care flavour composition, and/or
(iii.10) one or more oral care flavour ingredients having a spicy organoleptic attribute, wherein the total amount of oral care flavour ingredients having a spicy organoleptic attribute is at least 10 wt % of the oral care flavour composition, and/or
(iii.11) one or more oral care flavour ingredients having a sweet organoleptic attribute, wherein the total amount of oral care flavour ingredients having a sweet organoleptic attribute is at least 10 wt % of the oral care flavour composition.

10. The oral care flavour composition according to claim 6, wherein the at least one oral care flavour ingredient having another organoleptic attribute is selected from the group consisting of:
(iii.12) one or more oral care flavour ingredients having an eucalyptus organoleptic attribute selected from the group consisting of eucalyptus oil, eucalyptus oil reconsititutions, eucalyptol, and mixtures thereof; and/or
(iii.13) one or more oral care flavour ingredients having a spearmint organoleptic attribute selected from the group consisting of spearmint oil, spearmint oil reconstitutions, trans-carveol, cis-carveol, L-carvone, and mixtures thereof; and/or
(iii.14) one or more oral care flavour ingredients having a wintergreen organoleptic attribute selected from the group consisting of wintergreen oil, wintergreen oil reconsititutions, ethyl salicylate, methyl salicylate, and mixtures thereof; and/or
(iii.15) one or more oral care flavour ingredients having a herbal organoleptic attribute selected from the group consisting of camomile oil, camomile oil reconsititutions, juniper berry oil, juniper berry oil reconstitutions, lavandin oil, lavandin oil reconstitutions, lavender oil, lavender oil reconstitutions, rosemary oil, rosemary oil reconstitutions, sage oil, sage oil reconstitutions, thyme oil, thyme oil reconsititutions, thymol, and mixtures thereof; and/or
(iii.16) one or more oral care flavour ingredients having a citrus organoleptic attribute selected from the group consisting of bergamot oil, bergamot oil reconstitutions, citral, grapefruit oil, grapefruit oil reconstitutions, lemongrass oil, lemongrass oil reconstitutions, lemon oil, lemon oil reconstitutions, D-limonene, L-limonene, lime oil, lime oil reconstitutions, mandarin oil, mandarin oil reconstitutions, orange oil, orange oil reconstitutions, orange bitter oil, orange bitter oil reconstitutions, orange blood oil, orange blood oil reconstitutions, tangerine oil, tangerine oil reconsititutions, and mixtures thereof; and/or
(iii.17) one or more oral care flavour ingredients having a floral organoleptic attribute selected from the group consisting of citronellol, geranium oil, geranium oil reconstitutions, geranyl acetate, geraniol, hydroxycitronellal, ionone alpha, ionone beta, jasmin oil, jasmin oil reconstitutions, linalol, phenyl ethyl alcohol, a mixture of 3,7-dimethyloct-6-en-1-ol and 3,7-dimethyloct-7-en-1-ol, tetrahydro geraniol, rose oil, rose oil reconstitutions, terpineol alpha, ylang ylang oil, ylang ylang oil reconstitutions, and mixtures thereof; and/or
(iii.18) one or more oral care flavour ingredients having a fruity organoleptic attribute selected from the group consisting of cassyrane, cis-3-hexenyl acetate, damascenone, damascone beta, decalactone gamma, dimethyl sulphide, dodecalactone gamma, ethyl 2-methyl butyrate, ethyl acetate, ethyl butyrate, ethyl heptanoate, ethyl hexanoate, ethyl iso-butyrate, ethyl lactate, hexyl acetate, iso-amyl acetate, iso-amyl butyrate, iso-amyl propionate, raspberry ketone, trans-2-hexenal, and mixtures thereof; and/or
(iii.19) one or more oral care flavour ingredients having a cooling organoleptic attribute selected from the group consisting of N-(4-cyanomethylphenyl) p-menthane carboxamide, 2-isopropyl-5-methyl-N-(2-(pyridin-2-yl)ethyl)cyclohexane carboxamide, 3-(5-methyl-2-propan-2-ylcyclohexyl)oxypropane-1,2-diol, (1R,2R, 5S)-5-methyl-2-prop-1-en-2-ylcyclohexan-1-ol, ethyl 2-[[(1R,2S,5R)-5-methyl-2-propan-2-ylcyclohexanecarbonyl]amino]acetate, (1R,2S,5R)-N-(4-methoxyphenyl)-5-methyl-2-(1-methylethyl)cyclohexanecarboxamide, N-ethyl-5-methyl-2-propan-2-ylcyclohexane-1-carboxamide, N,2,3-trimethyl-2-propan-2-ylbutanamide, (2E)-3-(1,3-benzodioxol-5-yl)-N,N-diphenylacrylamide, 2-(p-tolyloxy)-N-(1H-pyrazol-5-yl)-N-((thiophen-2-yl)methyl)acetamide, [(1R,2S,5R)-5-methyl-2-propan-2-ylcyclohexyl]2-hydroxypropanoate, 2-isopropyl-5-methylcyclohexyloxycarbonyloxy-2-hydroxypropane, 2-hydroxypropyl (5-methyl-2-propan-2-ylcyclohexyl) carbonate, 9-methyl-6-(1-methylethyl)-1,4-dioxaspiro-[4,5 ]decan-2-methanol, menthyl lactate, mono-menthyl succinate, menthyl methyl ether, 1-methyl-3-hydroxybutyrate, 2-{[5-methyl-2-(propan-2-yl)cyclohexyl]oxy}ethanol, 2-(2-hydroxypropan-2-yl)-5-methylcyclohexan-1-ol, and mixtures thereof; and/or
(iii.20) one or more oral care flavour ingredients having a warming organoleptic attribute selected from the group consisting of capsaicine, capsicum oleoresin, gingerol, nonivamide, piperine, vanillyl butyl ether, and mixtures thereof; and/or
(iii.21) one or more oral care flavour ingredients having a spicy organoleptic attribute selected from the group consisting of cassia oil, cassia oil reconstitutions, cinnamic aldehyde, clove bud oil, clove bud oil reconstitutions, clove leaf oil, clove leaf oil reconstitutions, eugenol, ginger oil, ginger oil reconstitutions, ginger oleoresin, black pepper oil, black pepper oil reconstitutions, cardamom oil, cardamom oil reconstitutions, nutmeg oil, nutmeg oil reconstitutions, cinnamon leaf oil, cinnamon leaf oil reconstitutions, cinnamon bark oil, cinnamon bark oil reconstitutions, safraleine, and mixtures thereof; and/or
(iii.22) one or more oral care flavour ingredients having a sweet organoleptic attribute selected from the group consisting of acetyl methyl carbinol, benzaldehyde, cinnamic alcohol, decalactone delta, ethyl maltol, fenugreek oil, fenugreek oil reconstitutions, furaneol, hexalactone gamma, heliotropin, isobutavan, nonalactone gamma, octalactone gamma, p-methyl acetophenone, maltol, myrrh oil, myrrh oil reconstitutions, vanilla oil, vanilla oil reconstitutions, vanillin, and mixtures thereof.

11. An oral care consumer product comprising the oral care flavour composition according to claim 6 and a suitable base.

12. A method of enhancing the well-being of a human subject, the method comprising the step of contacting the human subject with the oral care composition according to claim 6.
